(19) United States
(12) Patent Application Publication
(10) Pub. No.: US 2001/0027788 A1
(43) Pub. Date: Oct. 11, 2001

(54) Computer for scuba diving

(76) Inventors: Giovanni Garofalo, Rapallo (IT); Claudio Ferrantino, Camogli (IT)

Correspondence Address: LARSON & TAYLOR, PLC, 1199 NORTH FAIRFAX STREET, SUITE 900, ALEXANDRIA, VA, 22314, US

(21) Appl. No.: 09/824,236

(22) Filed: Apr. 3, 2001

(30) Foreign Application Priority Data
Apr. 7, 2000 (IT) GE2000A000054

(51) Int. Cl. A62B 7/00; A62B 9/00; A61M 15/00; A61M 16/00; A62B 18/00

(52) U.S. Cl. 128/200.24; 128/205.22

(57) ABSTRACT

Computer for scuba diving consisting of at least a processor and of at least a manometer, both housed within a sheath, said manometer being connected to one or more bottles containing compressed air. The said processor includes a corresponding central unit for data processing, operatively connected to the manometer by means of connecting elements which allow said processor and said manometer to exchange information flows and also to be positioned within the housing sheath independently one from the other, so as to obtain a computer for scuba diving with a modular structure.

### BACKGROUND AND SUMMARY OF THE INVENTION

The present invention relates to computers used by scuba divers during diving sessions.

As is known, during a diving session with bottles the diver needs to know and check a given amount of data, among which the remaining pressure of the bottles, the remaining air time, the air consumption, the length of the decompression stages and others. To this purpose it is known about integrated computers for scuba diving carrying out, by means of suitable sensors, a series of checks on the parameters which are necessary for the calculation and the visualization on a suitable display of said data; for instance said integrated computers are connected by means of a pipe or intake to a pressure sensor placed on the first stage of pressure reduction at the output of the bottles. Said known computers, therefore, combine the functions of a processor provided with a central processing unit or CPU (Control Process Unit) with those of a traditional manometer integrated with said processing unit, at least partially separating the circuits. Said integration between the processing unit and the manometer involves some obvious constructing problems and turns the computer for scuba diving into a highly sophisticated device, extremely expensive to buy and maintain, since the diver has to buy both the processing unit and the manometer, said unit and said manometer being integrated and not to be separated, and moreover, in case the unit or the manometer get damaged, both have to be replaced.

The present invention, therefore, aims at carrying out a computer for scuba diving which, beyond being versatile and easy to build, allows the scuba diver to buy its constituting elements separately, that is to say either the processor or the manometer, and to mount them onto a single support even at different times, and which also enables, if either element gets damaged, the replacement of said element independently from the other element and from the rest of the computer.

Said aim is achieved by the present invention by means of a computer for scuba diving consisting of at least a processing unit and of at least a manometer, both being housed within a sheath, said manometer being connected to one or more bottles containing air under pressure; in said computer the processor consists of a corresponding central unit for data processing, which is operatively connected to the manometer by means of connecting elements allowing said processor and said manometer to exchange information flows and also to be positioned within the housing sheath independently one form the other, so as to obtain a computer for scuba diving with a modular structure.

Advantageously, therefore, by means of a computer for scuba diving carried out with a modular structure according to the present invention, the scuba diver can use a single sheath housing both the processor and the manometer, or either the processor or the manometer, allowing their separate purchase or an independent replacement in case of damage or breakdown.

### BRIEF DESCRIPTION OF THE DRAWINGS

Further aims and advantages of the present invention will be better understood in the following description, regarded as a mere non-limiting example, and referring to the enclosed drawings, in which:

FIG. 1 shows a view of a first form of embodiment of a computer for scuba diving according to the present invention;

FIG. 1a shows a block diagram referring to the first form of embodiment in FIG. 1 of the computer for scuba diving;

FIG. 2 shows a view of a second form of embodiment of the computer for scuba diving according to the present invention;

FIG. 2a shows a block diagram referring to the second form of embodiment in FIG. 2 of the computer;

FIG. 3 shows a view of a third form of embodiment of the computer according to the present invention;

FIG. 3a shows a block diagram referring to the third form of embodiment in FIG. 3 of the computer.

### DESCRIPTION OF THE PREFERRED EMBODIMENTS OF THE INVENTION

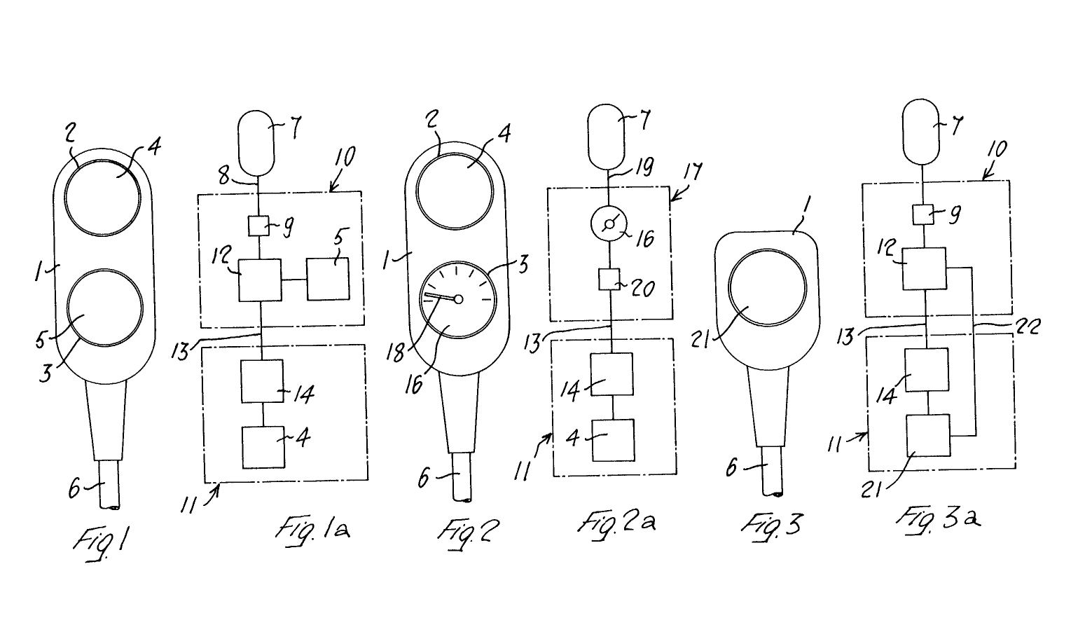

FIG. 1 shows a first form of embodiment of a computer for scuba diving according to the present invention. Said computer consists of a sheath 1 on which upper surface two sealed housings 2 and 3 are obtained, said housings being respectively meant for two digital displays 4 and 5: the display 4 belonging to a processor 11 and the display 5 belonging to an electronic manometer 10. An end of a pipe or intake 6 is fixed to the back portion of said sheath 1, the other end being connected to a first reducing stage at the output of a bottle 7, shown in the block diagram in FIG. 1a. Said diagram shows the above-mentioned bottle 7 connected by means of a wire 8 introduced into the intake 6 to a transducer 9 turning pressure signals into voltage signals. Said voltage signals are transferred to a central processing unit 12 belonging to the electronic manometer 10, which processes the data received from said transducer 9 and shows them on the display 5, for instance said data can refer to the remaining pressure within the bottle 7. The central unit 12 of the electronic manometer 10 is connected to a central unit 14 for data processing introduced into the processor 11 by means of a connecting element 13, which can be an IR-device communicating with corresponding transmission and reception means provided on the electronic manometer 10 and on the processor 11, a simple electric wire cable, a radio device or others. It is thus possible to carry out a computer for scuba diving in which the two main portions it consists of, that is to say, the processor 11 and the electronic manometer 10, are totally separable and autonomous and can be introduced into the sheath 1 at different times; then by means of the above-mentioned connecting element 13 it is possible to establish a continuous data flow between said portions, so that from the display 5 of the electronic manometer 10 it will be possible to select the visualization of a given set of parameters (for instance those referring to the bottle 7), while other parameters can be visualized on the display 4 of the processor 11 (for instance the air time, the air consumption, the decompression stages and others).

FIG. 2 shows a second form of embodiment of the present computer for scuba diving. As can be observed, the sheath 1 and the display 4 of the processor 11 are wholly similar to those in the form of embodiment described in FIG. 1. In this variant the electronic manometer 10 has been replaced by a mechanical manometer 17 provided with a sensitive element (FIG. 2a) connected on one end to the bottle 7 and on the other end to a pointer 18 rotatably fixed in the center of a dial 16 of said mechanical manometer 17. The deformation of the sensitive element 19 due to a pressure variation in the bottle 7 will be detected by the pointer 18 which will rotate on the dial 16 so as to allow the diver to read said pressure variation. As can be observed from the diagram in FIG. 2a, the moving signals of the pointer 18 are turned into voltage signals by a transducer 20 which communicates them to the central unit 14 for data processing of the processor 4 by means of a connecting element 13 previously described. The scuba diver can thus read, as with an ordinary manometer for scuba diving, the remaining pressure of the bottle 7 on the dial 16, and s/he can simultaneously read in real time the remaining air time on the display 4 of the processor 11, which has calculated said air time on the basis of the data received from the mechanical manometer 17. As in the previous form of embodiment, said display 4 can obviously also show other parameters which are useful to the scuba diver during the diving session.

FIGS. 3 and 3a show a third form of embodiment of the present computer. As can be observed, a single display 21 is provided on the sheath 1, said display being used by the central units 12 and 14 respectively belonging to an electronic manometer 10 and to a processor 11, the latter being wholly similar to those described in FIG. 1a. In this case the central unit 12 of the electronic manometer 10 is connected by means of a wire 22 to the display 21, on which it will be obviously possible to read all the data requested by the diver, such as bottle pressure, air time and so on. The use of a single display 21, therefore, involves the addition of another connection (the wire 22) between the manometer 10 and the processor 11, which nevertheless does not alter the modular structure of said computer, allowing in any case the diver to separate said processor 11 and manometer 10, and representing, where necessary, an improvement in terms of compactness on the diver's instruments.

1. Computer for scuba diving consisting of at least a processor and of at least a manometer, both housed within a sheath, said manometer being connected to one or more bottles containing compressed air, characterized in that the processor includes a corresponding central unit for data processing, operatively connected to the manometer by means of connecting elements which allow said processor and said manometer to exchange information flows and also to be positioned within the housing sheath independently one from the other, so as to obtain a computer for scuba diving with a modular structure.

2. Computer for scuba diving according to claim 1, characterized in that the surface of the said sheath is provided with one or more displays for the visualization of the data detected and processed by the said manometer and by the said processor.

3. computer for scuba diving according to claim 1, characterized in that the said manometer and the said processor are connected one to the other by means of optical connecting elements, such as an IR-device or others.

4. Computer for scuba diving according to claim 1, characterized in that the said manometer and the said processor are connected one to the other by means of electric connecting elements, such as one or more wires or others.

5. Computer for scuba diving according to claim 1, characterized in that the said manometer and the said processor are connected one to the other by means of wireless connecting elements.

6. Computer for scuba diving according to claim 1, characterized in that the said manometer is electronic and consists of a central unit for data processing connected to one or more bottles by means of a transducer and connected to a corresponding display for data visualization, said central unit of the said manometer being connected to the said central unit of the said processor, and said central unit of the said processor being connected to a corresponding display for data visualization.

7. Computer for scuba diving according to claim 6, characterized in that the said display of the said electronic manometer shows data such as the remaining pressure of the bottles, and the said display of the said processor shows data such as the remaining air time, the air consumption and further information which can be useful to a scuba diver during a diving session.

8. Computer for scuba diving according to claim 6, characterized in that the said data processing central units of the said electronic manometer and of the said processor are connected to a single display for data visualization.

9. Computer for scuba diving according to claim 1, characterized in that the said manometer is mechanical and consists of a dial provided with a pointer connected to a sensitive element for the pressure of the bottles, the pressure data detected by said mechanical manometer being transferred to the said central unit of the said processor by means of a transducer provided within the said manometer, and being visualized on a suitable display connected to the said central unit of the said processor.
